SEQUENCE LISTING (14 sequences)

SEQ ID NO: 1 - DNA, 21 bp, Artificial sequence
  Single strand DNA oligonucleotide
gagtagtggccctggttgctc

SEQ ID NO: 2 - DNA, 17 bp, Artificial sequence
  Single strand DNA oligonucleotide
cgctggtcgcttgccct

SEQ ID NO: 3 - DNA, 18 bp, Artificial sequence
  Single strand DNA oligonucleotide
ggtcctgagctgccggtc

SEQ ID NO: 4 - DNA, 3187 bp, Homo sapiens
aaagctctgggccccagggaggaggctcagcacagagagtggaaaacagcagaggtgacagagcagccgtgctcgaagcgttcctggagcccaagctctcctccacaggtgaagacagggccagcaggagacaccatggggcacctctcagccccacttcacagagtgcgtgtaccctggcaggggcttctgctcacagcctcacttctaaccttctggaacccgcccaccactgcccagctcactactgaatccatgccattcaatgttgcagaggggaaggaggttcttctccttgtccacaatctgccccagcaactttttggctacagctggtacaaaggggaaagagtggatggcaaccgtcaaattgtaggatatgcaataggaactcaacaagctaccccagggcccgcaaacagcggtcgagagacaatataccccaatgcatccctgctgatccagaacgtcacccagaatgacacaggattctacaccctacaagtcataaagtcagatcttgtgaatgaagaagcaactggacagttccatgtatacccggagctgcccaagccctccatctccagcaacaactccaaccctgtggaggacaaggatgctgtggccttcacctgtgaacctgagactcaggacacaacctacctgtggtggataaacaatcagagcctcccggtcagtcccaggctgcagctgtccaatggcaacaggaccctcactctactcagtgtcacaaggaatgacacaggaccctatgagtgtgaaatacagaacccagtgagtgcgaaccgcagtgacccagtcaccttgaatgtcacctatggcccggacacccccaccatttccccttcagacacctattaccgtccaggggcaaacctcagcctctcctgctatgcagcctctaacccacctgcacagtactcctggcttatcaatggaacattccagcaaagcacacaagagctctttatccctaacatcactgtgaataatagtggatcctatacctgccacgccaataactcagtcactggctgcaacaggaccacagtcaagacgatcatagtcactgataatgctctaccacaagaaaatggcctctcacctggggccattgctggcattgtgattggagtagtggccctggttgctctgatagcagtagccctggcatgttttctgcatttcgggaagaccggcagctcaggaccactccaatgacccacctaacaagatgaatgaagttacttattctaccctgaactttgaagcccagcaacccacacaaccaacttcagcctccccatccctaacagccacagaaataatttattcagaagtaaaaaagcagtaatgaaacctgtcctgctcactgcagtgctgatgtatttcaagtctctcaccctcatcactaggagattcctttcccctgtaggggtagaggggtggggacagaaacaactttctcctactcttccttcctaataggcatctccaggctgcctggtcactgcccctctctcagtgtcaatagatgaaagtacattgggagtctgtaggaaacccaaccttcttgtcattgaaatttggcaaagctgactttgggaaagagggaccagaacttcccctcccttccccttttcccaacctggacttgttttaaacttgcctgttcagagcactcattccttcccacccccagtcctgtcctatcactctaattcggatttgccatagccttgaggttatgtccttttccattaagtacatgtgccaggaaacaagagagagagaaagtaaaggcagtaatgccttctcctatttctccaaagccttgtgtgaactcaccaaacacaagaaaatcaaatatataaccaatagtgaaatgccacacctttgtccactgtcagggttgtctacctgtaggatcagggtctaagcaccttggtgcttagctagaataccacctaatccttctggcaagcctgtcttcagagaacccactagaagcaactaggaaaatcacttgccaaaatccaaggcaattcctgatggaaaatgcaaaagcacatatatgttttaatatctttatgggctctgttcaaggcagtgctgagagggaggggttatagcttcaggagggaaccagcttctgataaacacaatctgctaggaacttgggaaaggaatcagagagctgcccttcagcgattatttaaattattgttaaagaatacacaatttggggtattgggatttttctccttttctctgagacattccaccattttaatttttgtaactgcttatttatgtgaaaagggttatttttacttagcttagctatgtcagccaatccgattgccttaggtgaaagaaaccaccgaaatccctcaggtcccttggtcaggagcctctcaagattttttttgtcagaggctccaaatagaaaataagaaaaggttttcttcattcatggctagagctagatttaactcagtttctaggcacctcagaccaatcatcaactaccattctattccatgtttgcacctgtgcattttctgtttgcccccattcactttgtcaggaaaccttggcctctgctaaggtgtatttggtccttgagaagtgggagcaccctacagggacactatcactcatgctggtggcattgtttacagctagaaagctgcactggtgctaatgccccttggggaaatggggctgtgaggaggaggattataacttaggcctagcctcttttaacagcctctgaaatttatcttttcttctatggggtctataaatgtatcttataataaaaaggaaggacaggaggaagacaggcaaatgtacttctcacccagtcttctacacagatggaatctctttggggctaagagaaaggttttattctatattgcttacctgatctcatgttaggcctaagaggctttctccaggaggattagcttggagttctctatactcaggtacctctttcagggttttctaaccctgacacggactgtgcatactttccctcatccatgctgtgctgtgttatttaatttttcctggctaagatcatgtctgaattatgtatgaaaattattctatgtttttataataaaaataatatatcagacatcgaaaaaaaaaa

SEQ ID NO: 5 - DNA, 3240 bp, Homo sapiens
aaagctctgggccccagggaggaggctcagcacagagagtggaaaacagcagaggtgacagagcagccgtgctcgaagcgttcctggagcccaagctctcctccacaggtgaagacagggccagcaggagacaccatggggcacctctcagccccacttcacagagtgcgtgtaccctggcaggggcttctgctcacagcctcacttctaaccttctggaacccgcccaccactgcccagctcactactgaatccatgccattcaatgttgcagaggggaaggaggttcttctccttgtccacaatctgccccagcaactttttggctacagctggtacaaaggggaaagagtggatggcaaccgtcaaattgtaggatatgcaataggaactcaacaagctaccccagggcccgcaaacagcggtcgagagacaatataccccaatgcatccctgctgatccagaacgtcacccagaatgacacaggattctacaccctacaagtcataaagtcagatcttgtgaatgaagaagcaactggacagttccatgtatacccggagctgcccaagccctccatctccagcaacaactccaaccctgtggaggacaaggatgctgtggccttcacctgtgaacctgagactcaggacacaacctacctgtggtggataaacaatcagagcctcccggtcagtcccaggctgcagctgtccaatggcaacaggaccctcactctactcagtgtcacaaggaatgacacaggaccctatgagtgtgaaatacagaacccagtgagtgcgaaccgcagtgacccagtcaccttgaatgtcacctatggcccggacacccccaccatttccccttcagacacctattaccgtccaggggcaaacctcagcctctcctgctatgcagcctctaacccacctgcacagtactcctggcttatcaatggaacattccagcaaagcacacaagagctctttatccctaacatcactgtgaataatagtggatcctatacctgccacgccaataactcagtcactggctgcaacaggaccacagtcaagacgatcatagtcactgataatgctctaccacaagaaaatggcctctcacctggggccattgctggcattgtgattggagtagtggccctggttgctctgatagcagtagccctggcatgttttctgcatttcgggaagaccggcagggcaagcgaccagcgtgatctcacagagcacaaaccctcagtctccaaccacactcaggaccactccaatgacccacctaacaagatgaatgaagttacttattctaccctgaactttgaagcccagcaacccacacaaccaacttcagcctccccatccctaacagccacagaaataatttattcagaagtaaaaaagcagtaatgaaacctgtcctgctcactgcagtgctgatgtatttcaagtctctcaccctcatcactaggagattcctttcccctgtaggggtagaggggtggggacagaaacaactttctcctactcttccttcctaataggcatctccaggctgcctggtcactgcccctctctcagtgtcaatagatgaaagtacattgggagtctgtaggaaacccaaccttcttgtcattgaaatttggcaaagctgactttgggaaagagggaccagaacttcccctcccttccccttttcccaacctggacttgttttaaacttgcctgttcagagcactcattccttcccacccccagtcctgtcctatcactctaattcggatttgccatagccttgaggttatgtccttttccattaagtacatgtgccaggaaacaagagagagagaaagtaaaggcagtaatgccttctcctatttctccaaagccttgtgtgaactcaccaaacacaagaaaatcaaatatataaccaatagtgaaatgccacacctttgtccactgtcagggttgtctacctgtaggatcagggtctaagcaccttggtgcttagctagaataccacctaatccttctggcaagcctgtcttcagagaacccactagaagcaactaggaaaatcacttgccaaaatccaaggcaattcctgatggaaaatgcaaaagcacatatatgttttaatatctttatgggctctgttcaaggcagtgctgagagggaggggttatagcttcaggagggaaccagcttctgataaacacaatctgctaggaacttgggaaaggaatcagagagctgcccttcagcgattatttaaattattgttaaagaatacacaatttggggtattgggatttttctccttttctctgagacattccaccattttaatttttgtaactgcttatttatgtgaaaagggttatttttacttagcttagctatgtcagccaatccgattgccttaggtgaaagaaaccaccgaaatccctcaggtcccttggtcaggagcctctcaagattttttttgtcagaggctccaaatagaaaataagaaaaggttttcttcattcatggctagagctagatttaactcagtttctaggcacctcagaccaatcatcaactaccattctattccatgtttgcacctgtgcattttctgtttgcccccattcactttgtcaggaaaccttggcctctgctaaggtgtatttggtccttgagaagtgggagcaccctacagggacactatcactcatgctggtggcattgtttacagctagaaagctgcactggtgctaatgccccttggggaaatggggctgtgaggaggaggattataacttaggcctagcctcttttaacagcctctgaaatttatcttttcttctatggggtctataaatgtatcttataataaaaaggaaggacaggaggaagacaggcaaatgtacttctcacccagtcttctacacagatggaatctctttggggctaagagaaaggttttattctatattgcttacctgatctcatgttaggcctaagaggctttctccaggaggattagcttggagttctctatactcaggtacctctttcagggttttctaaccctgacacggactgtgcatactttccctcatccatgctgtgctgtgttatttaatttttcctggctaagatcatgtctgaattatgtatgaaaattattctatgtttttataataaaaataatatatcagacatcgaaaaaaaaaa

SEQ ID NO: 6 - DNA, 3333 bp, Homo sapiens
aaagctctgggccccagggaggaggctcagcacagagagtggaaaacagcagaggtgacagagcagccgtgctcgaagcgttcctggagcccaagctctcctccacaggtgaagacagggccagcaggagacaccatggggcacctctcagccccacttcacagagtgcgtgtaccctggcaggggcttctgctcacagcctcacttctaaccttctggaacccgcccaccactgcccagctcactactgaatccatgccattcaatgttgcagaggggaaggaggttcttctccttgtccacaatctgccccagcaactttttggctacagctggtacaaaggggaaagagtggatggcaaccgtcaaattgtaggatatgcaataggaactcaacaagctaccccagggcccgcaaacagcggtcgagagacaatataccccaatgcatccctgctgatccagaacgtcacccagaatgacacaggattctacaccctacaagtcataaagtcagatcttgtgaatgaagaagcaactggacagttccatgtatacccggagctgcccaagccctccatctccagcaacaactccaaccctgtggaggacaaggatgctgtggccttcacctgtgaacctgagactcaggacacaacctacctgtggtggataaacaatcagagcctcccggtcagtcccaggctgcagctgtccaatggcaacaggaccctcactctactcagtgtcacaaggaatgacacaggaccctatgagtgtgaaatacagaacccagtgagtgcgaaccgcagtgacccagtcaccttgaatgtcacctatggcccggacacccccaccatttccccttcagacacctattaccgtccaggggcaaacctcagcctctcctgctatgcagcctctaacccacctgcacagtactcctggcttatcaatggaacattccagcaaagcacacaagagctctttatccctaacatcactgtgaataatagtggatcctatacctgccacgccaataactcagtcactggctgcaacaggaccacagtcaagacgatcatagtcactgagagacagaatctcaccatgttacccaggctggactcgaactcctgggctcaagcaatcctcccatctgtttcccaaagtgctgagattacagataatgctctaccacaagaaaatggcctctcacctggggccattgctggcattgtgattggagtagtggccctggttgctctgatagcagtagccctggcatgttttctgcatttcgggaagaccggcagggcaagcgaccagcgtgatctcacagagcacaaaccctcagtctccaaccacactcaggaccactccaatgacccacctaacaagatgaatgaagttacttattctaccctgaactttgaagcccagcaacccacacaaccaacttcagcctccccatccctaacagccacagaaataatttattcagaagtaaaaaagcagtaatgaaacctgtcctgctcactgcagtgctgatgtatttcaagtctctcaccctcatcactaggagattcctttcccctgtaggggtagaggggtggggacagaaacaactttctcctactcttccttcctaataggcatctccaggctgcctggtcactgcccctctctcagtgtcaatagatgaaagtacattgggagtctgtaggaaacccaaccttcttgtcattgaaatttggcaaagctgactttgggaaagagggaccagaacttcccctcccttccccttttcccaacctggacttgttttaaacttgcctgttcagagcactcattccttcccacccccagtcctgtcctatcactctaattcggatttgccatagccttgaggttatgtccttttccattaagtacatgtgccaggaaacaagagagagagaaagtaaaggcagtaatgccttctcctatttctccaaagccttgtgtgaactcaccaaacacaagaaaatcaaatatataaccaatagtgaaatgccacacctttgtccactgtcagggttgtctacctgtaggatcagggtctaagcaccttggtgcttagctagaataccacctaatccttctggcaagcctgtcttcagagaacccactagaagcaactaggaaaatcacttgccaaaatccaaggcaattcctgatggaaaatgcaaaagcacatatatgttttaatatctttatgggctctgttcaaggcagtgctgagagggaggggttatagcttcaggagggaaccagcttctgataaacacaatctgctaggaacttgggaaaggaatcagagagctgcccttcagcgattatttaaattattgttaaagaatacacaatttggggtattgggatttttctccttttctctgagacattccaccattttaatttttgtaactgcttatttatgtgaaaagggttatttttacttagcttagctatgtcagccaatccgattgccttaggtgaaagaaaccaccgaaatccctcaggtcccttggtcaggagcctctcaagattttttttgtcagaggctccaaatagaaaataagaaaaggttttcttcattcatggctagagctagatttaactcagtttctaggcacctcagaccaatcatcaactaccattctattccatgtttgcacctgtgcattttctgtttgcccccattcactttgtcaggaaaccttggcctctgctaaggtgtatttggtccttgagaagtgggagcaccctacagggacactatcactcatgctggtggcattgtttacagctagaaagctgcactggtgctaatgccccttggggaaatggggctgtgaggaggaggattataacttaggcctagcctcttttaacagcctctgaaatttatcttttcttctatggggtctataaatgtatcttataataaaaaggaaggacaggaggaagacaggcaaatgtacttctcacccagtcttctacacagatggaatctctttggggctaagagaaaggttttattctatattgcttacctgatctcatgttaggcctaagaggctttctccaggaggattagcttggagttctctatactcaggtacctctttcagggttttctaaccctgacacggactgtgcatactttccctcatccatgctgtgctgtgttatttaatttttcctggctaagatcatgtctgaattatgtatgaaaattattctatgtttttataataaaaataatatatcagacatcgaaaaaaaaaa

SEQ ID NO: 7 - DNA, 3475 bp, Homo sapiens
aaagctctgggccccagggaggaggctcagcacagagagtggaaaacagcagaggtgacagagcagccgtgctcgaagcgttcctggagcccaagctctcctccacaggtgaagacagggccagcaggagacaccatggggcacctctcagccccacttcacagagtgcgtgtaccctggcaggggcttctgctcacagcctcacttctaaccttctggaacccgcccaccactgcccagctcactactgaatccatgccattcaatgttgcagaggggaaggaggttcttctccttgtccacaatctgccccagcaactttttggctacagctggtacaaaggggaaagagtggatggcaaccgtcaaattgtaggatatgcaataggaactcaacaagctaccccagggcccgcaaacagcggtcgagagacaatataccccaatgcatccctgctgatccagaacgtcacccagaatgacacaggattctacaccctacaagtcataaagtcagatcttgtgaatgaagaagcaactggacagttccatgtatacccggagctgcccaagccctccatctccagcaacaactccaaccctgtggaggacaaggatgctgtggccttcacctgtgaacctgagactcaggacacaacctacctgtggtggataaacaatcagagcctcccggtcagtcccaggctgcagctgtccaatggcaacaggaccctcactctactcagtgtcacaaggaatgacacaggaccctatgagtgtgaaatacagaacccagtgagtgcgaaccgcagtgacccagtcaccttgaatgtcacctatggcccggacacccccaccatttccccttcagacacctattaccgtccaggggcaaacctcagcctctcctgctatgcagcctctaacccacctgcacagtactcctggcttatcaatggaacattccagcaaagcacacaagagctctttatccctaacatcactgtgaataatagtggatcctatacctgccacgccaataactcagtcactggctgcaacaggaccacagtcaagacgatcatagtcactgagctaagtccagtagtagcaaagccccaaatcaaagccagcaagaccacagtcacaggagataaggactctgtgaacctgacctgctccacaaatgacactggaatctccatccgttggttcttcaaaaaccagagtctcccgtcctcggagaggatgaagctgtcccagggcaacaccaccctcagcataaaccctgtcaagagggaggatgctgggacgtattggtgtgaggtcttcaacccaatcagtaagaaccaaagcgaccccatcatgctgaacgtaaactataatgctctaccacaagaaaatggcctctcacctggggccattgctggcattgtgattggagtagtggccctggttgctctgatagcagtagccctggcatgttttctgcatttcgggaagaccggcagctcaggaccactccaatgacccacctaacaagatgaatgaagttacttattctaccctgaactttgaagcccagcaacccacacaaccaacttcagcctccccatccctaacagccacagaaataatttattcagaagtaaaaaagcagtaatgaaacctgtcctgctcactgcagtgctgatgtatttcaagtctctcaccctcatcactaggagattcctttcccctgtaggggtagaggggtggggacagaaacaactttctcctactcttccttcctaataggcatctccaggctgcctggtcactgcccctctctcagtgtcaatagatgaaagtacattgggagtctgtaggaaacccaaccttcttgtcattgaaatttggcaaagctgactttgggaaagagggaccagaacttcccctcccttccccttttcccaacctggacttgttttaaacttgcctgttcagagcactcattccttcccacccccagtcctgtcctatcactctaattcggatttgccatagccttgaggttatgtccttttccattaagtacatgtgccaggaaacaagagagagagaaagtaaaggcagtaatgccttctcctatttctccaaagccttgtgtgaactcaccaaacacaagaaaatcaaatatataaccaatagtgaaatgccacacctttgtccactgtcagggttgtctacctgtaggatcagggtctaagcaccttggtgcttagctagaataccacctaatccttctggcaagcctgtcttcagagaacccactagaagcaactaggaaaatcacttgccaaaatccaaggcaattcctgatggaaaatgcaaaagcacatatatgttttaatatctttatgggctctgttcaaggcagtgctgagagggaggggttatagcttcaggagggaaccagcttctgataaacacaatctgctaggaacttgggaaaggaatcagagagctgcccttcagcgattatttaaattattgttaaagaatacacaatttggggtattgggatttttctccttttctctgagacattccaccattttaatttttgtaactgcttatttatgtgaaaagggttatttttacttagcttagctatgtcagccaatccgattgccttaggtgaaagaaaccaccgaaatccctcaggtcccttggtcaggagcctctcaagattttttttgtcagaggctccaaatagaaaataagaaaaggttttcttcattcatggctagagctagatttaactcagtttctaggcacctcagaccaatcatcaactaccattctattccatgtttgcacctgtgcattttctgtttgcccccattcactttgtcaggaaaccttggcctctgctaaggtgtatttggtccttgagaagtgggagcaccctacagggacactatcactcatgctggtggcattgtttacagctagaaagctgcactggtgctaatgccccttggggaaatggggctgtgaggaggaggattataacttaggcctagcctcttttaacagcctctgaaatttatcttttcttctatggggtctataaatgtatcttataataaaaaggaaggacaggaggaagacaggcaaatgtacttctcacccagtcttctacacagatggaatctctttggggctaagagaaaggttttattctatattgcttacctgatctcatgttaggcctaagaggctttctccaggaggattagcttggagttctctatactcaggtacctctttcagggttttctaaccctgacacggactgtgcatactttccctcatccatgctgtgctgtgttatttaatttttcctggctaagatcatgtctgaattatgtatgaaaattattctatgtttttataataaaaataatatatcagacatcgaaaaaaaaaa

SEQ ID NO: 8 - DNA, 3528 bp, Homo sapiens
aaagctctgggccccagggaggaggctcagcacagagagtggaaaacagcagaggtgacagagcagccgtgctcgaagcgttcctggagcccaagctctcctccacaggtgaagacagggccagcaggagacaccatggggcacctctcagccccacttcacagagtgcgtgtaccctggcaggggcttctgctcacagcctcacttctaaccttctggaacccgcccaccactgcccagctcactactgaatccatgccattcaatgttgcagaggggaaggaggttcttctccttgtccacaatctgccccagcaactttttggctacagctggtacaaaggggaaagagtggatggcaaccgtcaaattgtaggatatgcaataggaactcaacaagctaccccagggcccgcaaacagcggtcgagagacaatataccccaatgcatccctgctgatccagaacgtcacccagaatgacacaggattctacaccctacaagtcataaagtcagatcttgtgaatgaagaagcaactggacagttccatgtatacccggagctgcccaagccctccatctccagcaacaactccaaccctgtggaggacaaggatgctgtggccttcacctgtgaacctgagactcaggacacaacctacctgtggtggataaacaatcagagcctcccggtcagtcccaggctgcagctgtccaatggcaacaggaccctcactctactcagtgtcacaaggaatgacacaggaccctatgagtgtgaaatacagaacccagtgagtgcgaaccgcagtgacccagtcaccttgaatgtcacctatggcccggacacccccaccatttccccttcagacacctattaccgtccaggggcaaacctcagcctctcctgctatgcagcctctaacccacctgcacagtactcctggcttatcaatggaacattccagcaaagcacacaagagctctttatccctaacatcactgtgaataatagtggatcctatacctgccacgccaataactcagtcactggctgcaacaggaccacagtcaagacgatcatagtcactgagctaagtccagtagtagcaaagccccaaatcaaagccagcaagaccacagtcacaggagataaggactctgtgaacctgacctgctccacaaatgacactggaatctccatccgttggttcttcaaaaaccagagtctcccgtcctcggagaggatgaagctgtcccagggcaacaccaccctcagcataaaccctgtcaagagggaggatgctgggacgtattggtgtgaggtcttcaacccaatcagtaagaaccaaagcgaccccatcatgctgaacgtaaactataatgctctaccacaagaaaatggcctctcacctggggccattgctggcattgtgattggagtagtggccctggttgctctgatagcagtagccctggcatgttttctgcatttcgggaagaccggcagggcaagcgaccagcgtgatctcacagagcacaaaccctcagtctccaaccacactcaggaccactccaatgacccacctaacaagatgaatgaagttacttattctaccctgaactttgaagcccagcaacccacacaaccaacttcagcctccccatccctaacagccacagaaataatttattcagaagtaaaaaagcagtaatgaaacctgtcctgctcactgcagtgctgatgtatttcaagtctctcaccctcatcactaggagattcctttcccctgtaggggtagaggggtggggacagaaacaactttctcctactcttccttcctaataggcatctccaggctgcctggtcactgcccctctctcagtgtcaatagatgaaagtacattgggagtctgtaggaaacccaaccttcttgtcattgaaatttggcaaagctgactttgggaaagagggaccagaacttcccctcccttccccttttcccaacctggacttgttttaaacttgcctgttcagagcactcattccttcccacccccagtcctgtcctatcactctaattcggatttgccatagccttgaggttatgtccttttccattaagtacatgtgccaggaaacaagagagagagaaagtaaaggcagtaatgccttctcctatttctccaaagccttgtgtgaactcaccaaacacaagaaaatcaaatatataaccaatagtgaaatgccacacctttgtccactgtcagggttgtctacctgtaggatcagggtctaagcaccttggtgcttagctagaataccacctaatccttctggcaagcctgtcttcagagaacccactagaagcaactaggaaaatcacttgccaaaatccaaggcaattcctgatggaaaatgcaaaagcacatatatgttttaatatctttatgggctctgttcaaggcagtgctgagagggaggggttatagcttcaggagggaaccagcttctgataaacacaatctgctaggaacttgggaaaggaatcagagagctgcccttcagcgattatttaaattattgttaaagaatacacaatttggggtattgggatttttctccttttctctgagacattccaccattttaatttttgtaactgcttatttatgtgaaaagggttatttttacttagcttagctatgtcagccaatccgattgccttaggtgaaagaaaccaccgaaatccctcaggtcccttggtcaggagcctctcaagattttttttgtcagaggctccaaatagaaaataagaaaaggttttcttcattcatggctagagctagatttaactcagtttctaggcacctcagaccaatcatcaactaccattctattccatgtttgcacctgtgcattttctgtttgcccccattcactttgtcaggaaaccttggcctctgctaaggtgtatttggtccttgagaagtgggagcaccctacagggacactatcactcatgctggtggcattgtttacagctagaaagctgcactggtgctaatgccccttggggaaatggggctgtgaggaggaggattataacttaggcctagcctcttttaacagcctctgaaatttatcttttcttctatggggtctataaatgtatcttataataaaaaggaaggacaggaggaagacaggcaaatgtacttctcacccagtcttctacacagatggaatctctttggggctaagagaaaggttttattctatattgcttacctgatctcatgttaggcctaagaggctttctccaggaggattagcttggagttctctatactcaggtacctctttcagggttttctaaccctgacacggactgtgcatactttccctcatccatgctgtgctgtgttatttaatttttcctggctaagatcatgtctgaattatgtatgaaaattattctatgtttttataataaaaataatatatcagacatcgaaaaaaaaaa

SEQ ID NO: 9 - protein, 493 aa, Homo sapiens
GRGDRAAVLEAFLEPKLSSTGEDRASRRHHGAPLSPTSQSACTLAGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVDLTCSTNDTGISIRWFFKNQSLPSSERMKLSQGNTTLSINPVKREDAGTYWCEVFNPISKNQSDPIMLNVNYNALPQENGLSPGAIAGIVIGVVALVALIAVALACFLHFGKTGSSGPLQ

SEQ ID NO: 10 - protein, 468 aa, Homo sapiens
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPGAIAGIVIGVVALVALIAVALACFLHFGKTGRTTPMTHLTR

SEQ ID NO: 11 - protein, 368 aa, Homo sapiens
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTDNALPQENGLSPGAIAGIVIGVVALVALIAVALACFLHFGKTGSSGPLQ

SEQ ID NO: 12 - protein, 430 aa, Homo sapiens
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTDNALPQENGLSPGAIAGIVIGVVALVALIAVALACFLHFGKTGRASDQRDLTEHKPSVSNHTQDHSNDPPNKMNEVTYSTLNFEAQQPTQPTSASPSLTATEIIYSEVKKQ

SEQ ID NO: 13 - protein, 461 aa, Homo sapiens
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTERQNLTMLPRLDSNSWAQAILPSVSQSAEITDNALPQENGLSPGAIAGIVIGVVALVALIAVALACFLHFGKTGRASDQRDLTEHKPSVSNHTQDHSNDPPNKMNEVTYSTLNFEAQQPTQPTSASPSLTATEIIYSEVKKQ

SEQ ID NO: 14 - protein, 526 aa, Homo sapiens
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPGAIAGIVIGVVALVALIAVALACFLHFGKTGRASDQRDLTEHKPSVSNHTQDHSNDPPNKMNEVTYSTLNFEAQQPTQPTSASPSLTATEIIYSEVKKQ

(19) United States
(12) Patent Application Publication
(10) Pub. No.: US 2012/0122122 A1
(43) Pub. Date: May 17, 2012

(54) METHOD OF DIAGNOSING CANCER

(75) Inventor: Gal Markel, Tel Aviv (IL)

(73) Assignee: TEL HASHOMER MEDICAL RESEARCH INFRASTRUCTURE AND SERVICES LTD., Ramat-Gun (IL)

(21) Appl. No.: 13/384,973

(22) PCT Filed: Jul. 21, 2010
(86) PCT No.: PCT/IL10/00581

§ 371 Date: Feb. 6, 2012

Related U.S. Application Data
(60) Provisional application No. 61/227,130, filed on Jul. 21, 2009.

(51) Int. Cl. G01N 33/574

(52) U.S. Cl. 435/7.24

(57) ABSTRACT

A method of diagnosing cancer is provided. The method comprising determining a level of CEACAM1 on isolated peripheral blood lymphocytes (PBLs) of a subject in need thereof, wherein an upregulation of the level of CEACAM1 above a predetermined threshold is indicative of cancer in said subject.

### RELATED APPLICATION/S

This application claims the benefit of priority under 35 USC 119(e) of U.S. Provisional Patent Application No. 61/227,130 filed Jul. 21, 2009, the contents of which are incorporated herein by reference in their entirety.

### FIELD AND BACKGROUND OF THE INVENTION

The present invention, in some embodiments thereof, relates to methods and kits for diagnosing cancer.

Cancer is a class of diseases in which a group of cells display uncontrolled growth (division beyond the normal limits), invasion (intrusion on and destruction of adjacent tissues), and sometimes metastasis. Cancer affects people at all ages with the risk for most types increasing with age. Cancer caused about 13% of all human deaths in 2007 (7.6 million).

There is a need for a rapid, economical and accurate diagnostic test for cancer.

The human Carcinoembryonic Ag (CEA) protein family encompasses several forms of proteins with different biochemical features. All CEA family genes have been classified into two major subfamilies, the CEA cell adhesion molecule (CEACAM) and the pregnancy-specific glycoprotein subgroups. The CEACAM proteins, which are part of the larger Ig superfamily, include CEACAM1, -3, -4, -5, -6, -7, and -8. They share a common basic structure of sequentially ordered different Ig-like domain(s) with considerable degree of homology. CEACAM5 is GPI-linked to cell surface, but it also appears in a soluble form in the peripheral blood where it is more recognized as the tumor marker CEA used to monitor colorectal cancer patients. CEACAM1 is a transmembrane protein that can be detected on some immune cells as well as on epithelial cells [Hammarström S (1999). Semin Cancer Biol 9: 67-81]. A striking association was observed between the presence of cell-bound CEACAM1 on primary cutaneous melanoma lesions and the development of metastatic disease with poor prognosis [Thies A et al. (2002) J Clin Oncol 20: 2530-2536]. The prognostic strength of melanoma associated CEACAM1 was similar or even superior to the widely accepted Breslow score [Thies et al. Supra]. Remarkably, a similar association was observed in lung adenocarcinoma specifically [Laack et al (2002) J Clin Oncol 20: 4279-4284] but also generally in non small cell lung cancers [Sienel et al (2003) Clin Cancer Res 9: 2260-2266].

Interestingly, the presence of human soluble CEACAM1 protein has been observed in the serum of healthy donors [Dráberová et al (2000) Immunology 101: 279-287; Kondo et al (2001). J Gastroenterol 36: 470-475; Svenberg et al. (1979) Clin Exp Immunol 36:317-325] and was found elevated in the sera of patients with biliary diseases including obstructive jaundice, primary biliary cirrhosis, autoimmune hepatitis and cholangiocarcinoma. Furthermore, it has been recently shown that serum CEACAM1 level is increased in some pancreatic adenocarcinoma patients [Simeone et al (2007) Pancreas 34: 436-443], presenting evidence for the potential role of soluble CEACAM1 as a tumor marker. Normally, circulating lymphocytes do not express CEACAM1 [Moller et al (1996) Int J Cancer 65: 740-745; Kammerer et al (1998) Eur J Immunol 28: 3664-3674], as it is upregulated on lymphocytes mainly following activation.

U.S. Application 20070071758 relates to methods for diagnosing cancer in vitro and in vivo using a CEACAM1 binding agent conjugated to a detectable moiety.

U.S. Application 20080108140 regards CEACAM1 as a biomarker for congenital CMV infection.

U.S. Application 20090181403 regards the utilization of CEACAM1 as a biomarker for cancer. In particular this application regards the detection and measurement of soluble CEACAM1 levels for the detection and diagnosis of melanoma.

### SUMMARY OF THE INVENTION

According to an aspect of some embodiments of the present invention there is provided a method of diagnosing cancer, the method comprising determining a level of CEACAM1 on isolated peripheral blood lymphocytes (PBLs) of a subject in need thereof, wherein an upregulation of the level of CEACAM1 above a predetermined threshold is indicative of cancer in the subject.

According to some embodiments of the invention, the cancer is melanoma.

According to some embodiments of the invention, the PBLs comprise T cells.

According to some embodiments of the invention, the PBLs comprise NK cells.

According to some embodiments of the invention, the determining a level of CEACAM1 on isolated peripheral blood lymphocytes (PBLs) is effected by FACS.

According to some embodiments of the invention, the cancer does not include a hematological cancer.

According to some embodiments of the invention, the method further comprising informing the subject on the presence or absence of the cancer or stage thereof.

According to some embodiments of the invention, the method further comprising validating the diagnosis or prognosis using a method selected from the group consisting of surgical biopsy, imaging, pathology and molecular testing.

According to some embodiments of the invention, the determining is effected ex vivo.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-D are graphs illustrating high expression of CEACAM1 on circulating lymphocytes among melanoma patients. A scatter distribution of CEACAM1 expression profile on circulating NK cells is shown in FIG. 1a or T cells in FIG. 1b. There are three main groups of samples, healthy donors (black squares), patients with no evidence of disease (NED, black upright triangles) and patients with evidence of disease (WED, black inverse triangles). Each individual shape represents a single sample from the same group. Y axis denotes the percent of CEACAM1-positive circulating lymphocytes. (FIGS. 1c-d) WED patients were further categorized into patients that died of disease (DOD) during follow up and patients that remained alive with disease (AWD). Figure shows CEACAM1 scatter distribution on circulating NK cells (FIG. 1c) or T cells (FIG. 1d) in these subgroups. Horizontal lines indicate the median value of the group. Non parametric two sided t-test was used to compare between different groups, as indicated in each plot. * denotes P value<0.05, ** denotes P value<0.01 and *** denotes P value<0.001.

FIGS. 2A-C are graphs showing enhanced CEACAM1 functional expression that inhibits NK killing activity. FIG. 2a—Plots show the CEACAM1 expression profile on gated peripheral blood NK cells. Samples were derived either from healthy donors or melanoma patients, as indicated in the figure. FIG. 2b—Peripheral blood lymphocytes were tested for natural killing activity against NK-sensitive 221 cells. Target cells were either mock transfected (221/Mock—black bars) or stable transfected with CEACAM1 cDNA (221/CEACAM1—gray bars). Effector-to-target ratio was 50-to-1. Y-axis denotes the percent of specific lysis of target cells. Figure shows a representative experiment out of 3 performed. * denotes P value<0.05 FIG. 2c Peripheral blood lymphocytes derived from a healthy donor were cultured either in culture medium or in serum. Serum was derived either from an allogeneic healthy donor or from melanoma patients with either low or high percentage of CEACAM1-positive lymphocytes, as indicated in the figure. Peripheral blood lymphocytes from four different donors were tested, each in three different sera samples from each category. The figures show the staining results of gated lymphocytes of a representative experiment.

FIG. 3 illustrate dysregulated expression of NK activating receptors on circulating NK cells. The figure shows scatter distribution of various NK activating receptors (indicated in the figure) expression profile on circulating NK cells. There are three main groups of samples, healthy donors (black squares), patients with no evidence of disease (NED, black upright triangles) and patients with evidence of disease (WED, black inverse triangles). Each individual shape represents a single sample from the same group. Y axis denotes the percent of receptor-positive circulating NK cells. Horizontal lines indicate the median value of the group, which is indicated numerically below. Non parametric two sided t-test was used to compare between different groups, as indicated in each plot. * denotes P value<0.05, ** denotes P value<0.01.

### DESCRIPTION OF SPECIFIC EMBODIMENTS OF THE INVENTION

The present invention, in some embodiments thereof, relates to methods and kits for diagnosing cancer.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The American Cancer Society estimates the lifetime risk that an individual will develop cancer is 1 in 2 for men and 1 in 3 for women. The development of cancer, while still not completely understood, can be enhanced as a result of a variety of risk factors. For example, exposure to environmental factors (e.g., tobacco smoke) might trigger modifications in certain genes, thereby initiating cancer development. Alternatively, these genetic modifications may not require an exposure to environmental factors to become abnormal. Indeed, certain mutations (e.g., deletions, substitutions, etc.) can be inherited from generation to generation, thereby imparting an individual with a genetic predisposition to develop cancer.

Currently, the survival rates for many cancers are on the rise. One reason for this success is improvement in the detection of cancer at a stage at which treatment can be effective. Indeed, it has been noted that one of the most effective means to survive cancer is to detect its presence as early as possible. According to the American Cancer Society, the relative survival rate for many cancers would increase by about 15% if individuals participated in regular cancer screenings. Therefore, it is becoming increasingly useful to develop novel diagnostic tools to detect the cancer preferably before it develops or at an as early stage of development as possible.

Soluble CEACAM1 has been previously suggested as a tumor marker, especially for melanoma. Interestingly, soluble CEACAM1 is also apparent in the serum of healthy individuals albeit at lower levels.

The present inventor has uncovered that CEACAM1 is expressed on peripheral blood lymphocytes (PBLs) of tumor affected individuals, while expression of this protein on PBLs of healthy individuals is missing (undetectable using FACS).

This finding suggests that testing CEACAM1 expression on isolated lymphocytes can be effectively used in diagnosing cancer with reduced erroneously positive readings.

Thus, according to an aspect of the invention there is provided a method of diagnosing cancer, the method comprising determining a level of CEACAM1 on isolated peripheral blood lymphocytes (PBLs) of a subject in need thereof, wherein an upregulation of said level of CEACAM1 above a predetermined threshold is indicative of cancer in said subject.

As used herein the term “diagnosing” refers to determining presence or absence of a pathology, classifying a pathology or a symptom, determining a severity of the pathology, monitoring pathology progression, forecasting an outcome of a pathology and/or prospects of recovery. The term diagnosis also refers, in some embodiments thereof, to screening. Screening for cancer can lead to earlier diagnosis in specific cases. Early diagnosis may lead to extended life.

Non-limiting examples of cancers which can be diagnosed by the method of the invention include melanoma, intraocular melanoma, neoplasms of the central nervous system, tumors of the gastrointestinal tract (colon cancer, rectum cancer, anal region cancer, colorectal cancer, small and/or large bowel cancer, esophageal cancer, stomach cancer, pancreatic cancer, gastric cancer, small intestine cancer, adenocarcinoma arising in the small intestine, carcinoid tumors arising in the small intestine, lymphoma arising in the small intestine, mesenchymal tumors arising in the small intestine, gastrointestinal stromal tumors), gallbladder carcinoma, Biliary tract tumors, prostate cancer, kidney (renal) cancer (e.g., Wilms' tumor), liver cancer (e.g., hepatoblastoma, hepatocellular carcinoma), hepatobiliary cancer, biliary tree cancer, tumors of the Gallbladder, bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian cancer, cervical cancer, cancer of the vagina, cancer of the Vulva, lung cancer (e.g., small-cell and non-small cell lung carcinoma), nasopharyngeal, breast cancer, squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B cell, Burkitt, cutaneous T cell, histiocytic, lymphoblastic, T cell, thymic, cutaneous T-cell lymphoma, primary central nervous system lymphoma), gliomas, medullary thyroid carcinoma, testicular cancer, brain and head/neck cancer, gynecologic cancer, endometrial cancer, germ cell tumors, mesenchymal tumors, neurogenic tumors, cancer of the bladder, cancer of the ureter, cancer of the renal pelvis, cancer of the urethra, cancer of the penis, cancer of the testis, cancers of the uterine body, endometrial carcinoma, uterine sarcoma, peritoneal carcinoma and Fallopian Tube carcinoma, germ cell tumors of the ovary, sex cord-stromal tumors, cancer of the endocrine system, thyroid tumors, medullary thyroid carcinoma, thyroid lymphoma, parathyroid tumors, adrenal tumors, pancreatic endocrine tumors, sarcomas of the soft tissue and bone, benign and malignant mesothelioma, malignant peritoneal mesothelioma, malignant mesothelioma of the Tunica Vaginalis Testis, malignant mesothelioma of the Pericardium, skin cancer, cutaneous medulloblastomas, meningiomas, peripheral nerve tumors, Pineal region tumors, pituitary adenomas, craniopharyngiomas, acoustic neuromas, Glomus Jugulare tumors, Chordomas and Chondrosarcomas, Hemangioblastomas, Choroid Plexus Papillomas and Carcinomas, and spinal axis tumors.

According to a specific embodiment of the invention the cancer is not a hematological malignancy (cancer).

According to a specific embodiment of the invention the cancer is melanoma.

As used herein the phrase “subject in need thereof” refers to a human subject who is at risk of having cancer [e.g., a genetically predisposed subject, a subject with medical and/or family history of cancer, a subject who has been exposed to carcinogens, occupational hazard, environmental hazard] and/or a subject who exhibits suspicious clinical signs of cancer [e.g., blood in the stool or melena, unexplained pain, sweating, unexplained fever, unexplained loss of weight up to anorexia, changes in bowel habits (constipation and/or diarrhea), tenesmus (sense of incomplete defecation, for rectal cancer specifically), anemia and/or general weakness], changes to the shape or color of existing moles, itching, bleeding or ulcerating moles. Additionally or alternatively, the subject in need thereof can be a healthy human subject undergoing a routine well-being check up.

As used herein the term “CEACAM1” refers to the mRNA or protein product of the CEACAM1 gene e.g., Nucleotide sequences of CEACAM1 include, but are not limited to, NM_{—}001184816.1 GI:296317313 (SEQ ID NO: 5), NM_{—}001184813.1 GI:296317304 (SEQ ID NO: 6), NM_{—}001184815.1 GI:296317311 (SEQ ID NO: 7), NM_{—}001024912.2 GI:296317301 (SEQ ID NO: 8), NM_{—}001712.4 GI:296317298 (SEQ ID NO: 9). Protein sequences of CEACAM1 include but are not limited to, AAH24164.1 (SEQ ID NO: 10), AAH14473.1 (SEQ ID NO: 11), NP_{—}001171745.1 (SEQ ID NO: 12), NP_{—}001171742.1 (SEQ ID NO: 13), NP_{—}001171744.1 (SEQ ID NO: 14), P13688.2 (SEQ ID NO: 15).

As used herein, the phrase “peripheral blood lymphocytes” refers to a sample taken from circulating blood as opposed to blood cells sequestered within the lymphatic system, spleen, liver, or bone marrow. The term refers to large granular lymphocytes and small lymphocytes. Large granular lymphocytes include natural killer cells (NK cells). Small lymphocytes consist of T cells and B cells.

As used herein the term “isolated” refers to isolated from the natural environment. According to a specific embodiment, the term relates to serum purified i.e., no plasma.

Peripheral blood cell samples are typically taken using a syringe with a needle.

Methods of processing peripheral blood cell samples are known in the art and further described in the Examples section herein below.

It will be appreciated that determining the level of CEACAM1 in peripheral blood can be performed ex vivo (on a sample derived from the subject) as well as in vivo (within the subject).

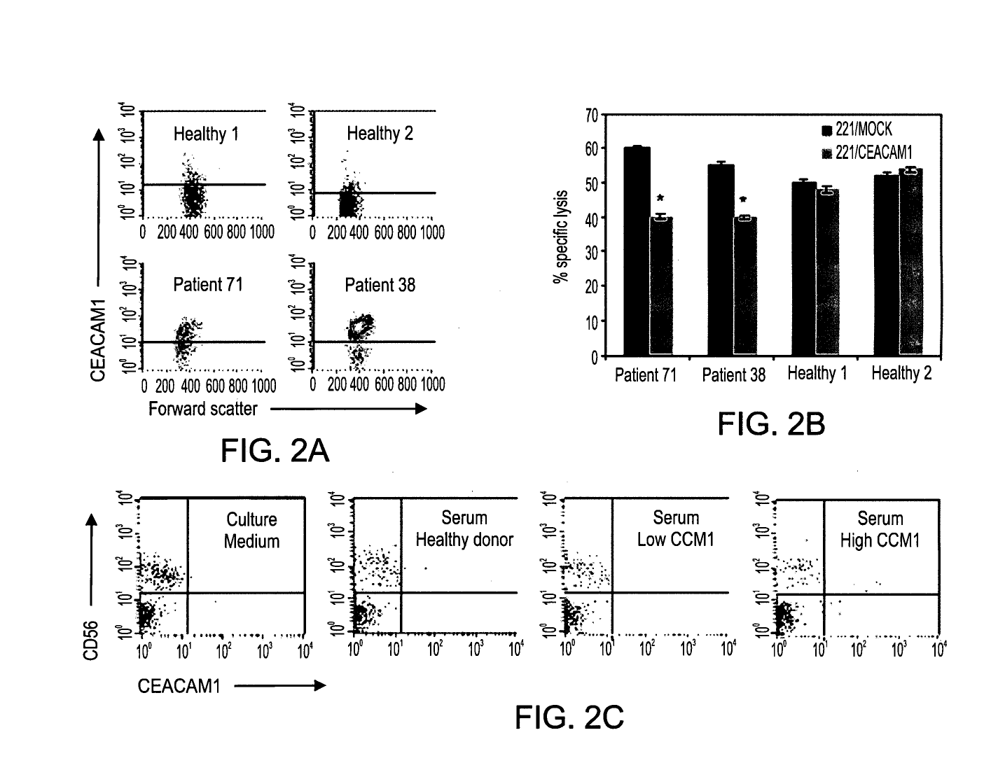

As used herein, the phrase “level of CEACAM1” refers to the degree of gene expression and/or gene product activity (e.g., inhibition of NK killing activity as shown in FIGS. 2A-C) of the CEACAM1 gene in the biological sample. Accordingly, the level of CEACAM1 can be determined at the amino acid level using protein detection methods.

Thus, the level of the CEACAM1 amino acid sequence (CEACAM1 protein) can be determined using a CEACAM1 specific antibody via the formation of an immunocomplex [i.e., a complex formed between the CEACAM1 antigen (a CEACAM1 amino acid sequence) present in the biological sample and the CEACAM1 specific antibody].

The immunocomplex of the present invention can be formed at a variety of temperatures, salt concentration and pH values which may vary depending on the method and the biological sample used and those of skills in the art are capable of adjusting the conditions suitable for the formation of each immunocomplex.

The term “antibody” as used in this invention includes intact molecules as well as functional fragments thereof, such as Fab, F(ab′)2, Fv or single domain molecules such as VH and VL to an epitope of an antigen. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; (5) Single chain antibody (“SCA”), a genetically engineered molecule containing the variable region of the light chain and the variable region of the heavy chain, linked by a suitable polypeptide linker as a genetically fused single chain molecule; and (6) Single domain antibodies are composed of a single VH or VL domains which exhibit sufficient affinity to the antigen.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (scFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing scFvs are described, for example, by Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10,: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

According to the method of this aspect of the present invention, an amount of immunocomplex formation is indicative of a diagnosis of the cancer. Various methods can be used to detect the formation of the CEACAM1 immunocomplex of the present invention and those of skills in the art are capable of determining which method is suitable for each immunocomplex.

Anti CEACAM1 antibodies are known in the art, some of which are described in the Examples section which follows (see also U.S. Pat. Application 20090181403, Which teaches polyclonal or monoclonal antibodies for CEACAM1, herein incorporated by reference in its entirety). Exemplar CEACAM1 specific antibodies include: Mouse monoclonal [29H2] to CEACAM1; Mouse monoclonal [GM8G5] to CEACAM1 (GM8G5 recognizes the Human CEACAM1 A2 domain); CEACAM1 antibody number 2037.00.02 (from Strategic Diagnostics Inc) binding CEACAM1 amino acids 35-134; the CEACAM1-specific antibody 4D1/C2; anti-CEACAM1 5F4 mAb; anti-CEACAM1 Kat4c mAb; or any combination or derivative thereof. The CEACAM1 binding agent can also be a member of the CEA protein family. Another antibody which can be used in accordance with the present teachings is such one that has the CDRs of the antibody produced from the hybridoma cell which has been deposited under ATCC Accession Number PTA-9974.

The CEACAM1 antibody used in the immunocomplex of the present invention can be labeled using methods known in the art. It will be appreciated that the labeled antibodies can be either primary antibodies (i.e., which bind to the specific antigen, e.g., a CEACAM1-specific antigen) or secondary antibodies (e.g., labeled goat anti rabbit antibodies, labeled mouse anti human antibody) which bind to the primary antibodies. The antibody can be directly conjugated to a label or can be conjugated to an enzyme.

Antibodies of the present invention can be fluorescently labeled (using a fluorescent dye conjugated to an antibody), radiolabeled (using radiolabeled e.g., ¹²⁵I, antibodies), or conjugated to an enzyme (e.g., horseradish peroxidase or alkaline phosphatase) and used along with a chromogenic substrate to produce a colorimetric reaction. The chromogenic substrates utilized by the enzyme-conjugated antibodies of the present invention include, but are not limited to, AEC, Fast red, ELF-97 substrate [2-(5′-chloro-2-phosphoryloxyphenyl)-6-chloro-4(3H)-quinazolinone], p-nitrophenyl phosphate (PNPP), phenolphthalein diphosphate, and ELF 39-phosphate, BCIP/INT, Vector Red (VR), salmon and magenta phosphate (Avivi C., et al., 1994, J Histochem. Cytochem. 1994; 42: 551-4) for alkaline phosphatase enzyme and Nova Red, diaminobenzidine (DAB), Vector(R) SG substrate, luminol-based chemiluminescent substrate for the peroxidase enzyme. These enzymatic substrates are commercially available from Sigma (St Louis, Mo., USA), Molecular Probes Inc. (Eugene, Oreg., USA), Vector Laboratories Inc. (Burlingame, Calif., USA), Zymed Laboratories Inc. (San Francisco, Calif., USA), Dako Cytomation (Denmark).

Detection of the CEACAM1 immunocomplex in PBCs can be performed using fluorescence activated cell sorting (FACS), enzyme linked immunosorbent assay (ELISA), Western blot and radio-immunoassay (RIA) analyses, immunoprecipitation (IP) with optionally the use of magnetic beads or by a molecular weight-based approach.

For Western blot the proteins are extracted from a cell sample and are subjected to electrophoresis (e.g., SDS-PAGE) and blotting to a membrane (e.g., nitrocellulose or PVDF). The membrane is then interacted with a CEACAM1 antibody which can be either directly labeled or further subjected to a secondary labeled antibody. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

In case the concentration of the antigen in the biological sample is low, detection of the antigen (CEACAM1 amino acid sequence) can be performed by immunoprecipitation (IP). For immunoprecipitation analysis the CEACAM1 antibody may directly interact with a sample (e.g., cell lysate) including CEACAM1 and the formed complex can be further detected using a secondary antibody conjugated to beads (e.g., if the CEACAM1 antibody is a mouse monoclonal antibody, the secondary antibody may be an anti-mouse antibody conjugated to e.g., Sepharose beads). The beads can be then precipitated by centrifugation, following which the precipitated proteins (e.g., CEACAM1 and anti CEACAM1 antibodies) can be detached from the beads (e.g., using denaturation at 95° C.) and further subjected to Western blot analysis using the CEACAM1 specific antibodies. Alternatively, the anti-CEACAM1 antibody and the beads-conjugated secondary antibody may be added to the biological sample containing the antigen (CEACAM1) to thereby form an immunocomplex, followed by Western blot analysis with anti-CEACAM1 antibodies.

FACS analysis enables the detection of antigens present on cell membranes such as CEACAM1. Briefly, CEACAM1 specific antibodies are linked to fluorophores and detection is performed by means of a cell sorting machine which reads the wavelength of light emitted from each cell as it passes through a light beam. This method may employ two or more antibodies simultaneously.

The level of CEACAM1 can be also determined using ELISA. Briefly, a sample containing CEACAM1 antigen is fixed to a surface such as a well of a microtiter plate. An antigen specific antibody (a CEACAM1 antibody) coupled to an enzyme is applied and allowed to bind to the antigen. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

The level of CEACAM1 can be also determined using radio-immunoassay (RIA). In one version, this method involves precipitation of the desired antigen (CEACAM1) with a specific antibody and radiolabeled antibody binding protein (e.g., protein A labeled with I¹²⁵) immobilized on a precipitable carrier such as agarose beads. The number of counts in the precipitated pellet is proportional to the amount of antigen.

In an alternate version of the RIA, a labeled antigen and an unlabeled antibody binding protein are employed. A sample containing an unknown amount of antigen is added in varying amounts. The decrease in precipitated counts from the labeled antigen is proportional to the amount of antigen in the added sample.

The level of CEACAM1 can be also determined using molecular weight-based approach. Since the immunocomplex exhibits a higher molecular weight than its components, methods capable of detecting such a change in the molecular weight can be also employed. For example, the immunocomplex can be detected by a gel retardation assay. Briefly, a non-denaturing acrylamide gel is loaded with samples. A shift in the size (molecular weight) of the protein product as compared with its components is indicative of the presence of an immunocomplex. Such a shift to a higher molecular weight can be viewed using a non-specific protein staining such as silver stain or Commassie blue stain.

It will be appreciated that analyzing an amount of CEACAM1 in PBCs may also be effected at the polynucleotide level. RNA detection methods can be performed using an isolated polynucleotide (e.g., a polynucleotide probe, an oligonucleotide probe/primer) capable of hybridizing to a CEACAM1 nucleic acid sequence such as the CEACAM1 transcript set forth by NM_{—}001184816.1 GI:296317313 (SEQ ID NO: 5), NM_{—}001184813.1 GI:296317304 (SEQ ID NO: 6), NM_{—}001184815.1 GI:296317311 (SEQ ID NO: 7), NM_{—}001024912.2 GI:296317301 (SEQ ID NO: 8), NM_{—}001712.4 GI:296317298 (SEQ ID NO: 8). Examples for such oligonucleotide probe/primer sequences are set forth in SEQ ID NOs:1-3. Such a polynucleotide can be at any size, such as a short polynucleotide (e.g., of 15-200 bases), an intermediate polynucleotide of 100-2000 bases and a long polynucleotide of more than 2000 bases.

The isolated polynucleotide probe used by the present invention can be any directly or indirectly labeled RNA molecule [e.g., RNA oligonucleotide (e.g., of 17-50 bases), an in vitro transcribed RNA molecule], DNA molecule (e.g., oligonucleotide, e.g., 15-50 bases, cDNA molecule, genomic molecule) and/or an analogue thereof [e.g., peptide nucleic acid (PNA)] which is specific to the CEACAM1 RNA transcript of the present invention.

Oligonucleotides designed according to the teachings of the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

The isolated polynucleotide used by the present invention can be labeled either directly or indirectly using a tag or label molecule. Such labels can be, for example, fluorescent molecules (e.g., fluorescein or Texas Red), radioactive molecule (e.g., ³²P-γ-ATP or ³²P-α-ATP) and chromogenic substrates [e.g., Fast Red, BCIP/INT, available from (ABCAM, Cambridge, Mass.)]. Direct labeling can be achieved by covalently conjugating a label molecule to the polynucleotide (e.g., using solid-phase synthesis) or by incorporation via polymerization (e.g., using an in vitro transcription reaction or random-primed labeling). Indirect labeling can be achieved by covalently conjugating or incorporating to the polynucleotide a non-labeled tag molecule (e.g., Digoxigenin or biotin) and subsequently subjecting the polynucleotide to a labeled molecule (e.g., anti-Digoxigenin antibody or streptavidin) capable of specifically recognizing the non-labeled tag.

The above-described polynucleotides can be employed in a variety of RNA detection methods such as Northern blot analysis, reverse-transcribed PCR (RT-PCR) [e.g., a semi-quantitative RT-PCR, quantitative RT-PCR using e.g., the Light Cycler™ (Roche)], RNA in situ hybridization (RNA-ISH), in situ RT-PCR stain [e.g., as described in Nuovo G J, et al. 1993, Intracellular localization of polymerase chain reaction (PCR)-amplified hepatitis C cDNA. Am J Surg Pathol. 17: 683-90, and Komminoth P, et al. 1994, Evaluation of methods for hepatitis C virus detection in archival liver biopsies. Comparison of histology, immunohistochemistry, in situ hybridization, reverse transcriptase polymerase chain reaction (RT-PCR) and in situ RT-PCR. Pathol Res Pract., 190: 1017-25] and oligonucleotide microarray analysis [e.g., using the Affymetrix microarray (Affymetrix®, Santa Clara, Calif.)].

As mentioned, a level of CEACAM1 in a PBC sample above a predetermined threshold is indicative of the cancer.

The “predetermined threshold” may be experimentally determined by comparing normal PBC samples (e.g., samples obtained from healthy subjects, not affected with cancer) to PBC samples derived from subjects known to have carcinogenesis such as CRC. Preferably, a statistically significant number of samples are analyzed.

It will be appreciated that the presence of the cancer can be further validated using additional assays. For example, in case the level of CEACAM1 detected in a PBC sample of a subject is above a predetermined threshold, additional assays such as Gold-standard assays including but not limited to histology, imaging, molecular markers, blood tests e.g., colon endoscopy followed by histological evaluations (including CEACAM1 immunostaining), the “ABCDE” and skin biopsy for melanoma, may be performed on the identified adenomas (in case adenomas are present).

Once results are obtained the subject is informed of the test results i.e., presence or absence of cancer and suitable treatments may be initiated.

Diagnostic compositions of the present invention may, if desired, be presented in an article of manufacture e.g., kit, such as an FDA approved kit, which may contain diagnostic reagents and instructions for use. The kit may also be accommodated by a notice associated with the container in a form prescribed by a governmental agency regulating the manufacture, use or sale of pharmaceuticals, which notice is reflective of approval by the agency of the form of the compositions or human or veterinary use.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Experimental Procedures

Patients and Controls

Patients with pathologically verified cutaneous malignant melanoma in all AJCC stages of disease were included. There were no exclusion criteria. Patients were broadly categorized clinically into two groups: a) patients with no evidence of disease (NED) at the time of blood sampling that were further subdivided into low risk of recurrence (AJCC stage I or II) and high risk of recurrence (AJCC stage III or IV); b) patients with evidence of active disease (WED) at the time of blood sampling, who were subcategorized according AJCC criteria. High risk NED patients (AJCC stages III and IV) have received prior therapy that yielded disease regression. All normal controls were in excellent health at the time of the study. All melanoma patients and healthy volunteers gave written informed consent prior to their participation in this study. This study was approved by the Sheba Medical Center Institutional Review Board.

Specimen Characteristics

Blood samples were obtained from healthy individuals and patients by veno-puncture (>3 ml)and standard handling procedures. Peripheral blood lymphocytes were purified using a density gradient and deep frozen in liquid nitrogen. Anonymous samples (marked only with ID number) were linked only to clinical-pathological data.

Study Design

Blood was obtained in the melanoma clinic with no case selection. None of the patients underwent surgery near the time of blood sampling. Study was retrospective: a single blood sample was obtained from all patients, frozen and analyzed at a later, technically convenient, point. Each sample was tested two independent times in triplicate repeats. Follow up in this study began from time of blood sampling. The mean follow up time was 12 months all groups, except for stage IV-M1c WED patients, due to death of some of the patients shortly after blood sampling (Table 1). The clinical endpoints examined were disease free period (DFP) for NED patients and survival for WED patients.

Antibodies

Antibodies directed against CEACAM proteins included in this work were: murine anti-human CEACAM1 monoclonal antibodies NC8 [Albarran-Somoza B, Franco-Topete R, Delgado-Rizo V et al (2006) CEACAM1 in cervical cancer and precursor lesions: association with human papillomavirus infection. J Histochem Cytochem. 54: 1393-1399], murine anti-human CEACAM1, 5, 6, 8 monoclonal antibody Kat4c (Dako, Glustrup Denmark) and purified rabbit polyclonal anti-human CEACAM1, 5, 6 antibodies (Dako, Glustrup Denmark). The following conjugated monoclonal antibodies were used: anti human CD3-FITC (IQ); anti human CD56-PE/Cy5.5 (eBiocience); anti human NKp46-APC (eBiocience); anti human NKp30-APC (eBiocience); anti human CD16-PE (eBiocience); anti human NKG2D-APC (R&D Systems, Minneapolis, Minn., USA), biotinylated NC8 and biotinylated rabbit polyclonal anti-human CEACAM1, 5, 6 antibodies. Secondary reagents included PE-conjugated F(ab′)2 fragments of goat anti human-Fc IgG (Jackson ImmunoResearch); FITC-conjugated F(ab′)2 fragments of goat anti mouse-Fc IgG (ICN) and PE-conjugated streptavidin (Jackson Immunoresearch). Biotinylation of antibodies was performed with SS biotin (Chemicon) according to manufacturer's instructions.

Flow Cytometry

Binding of antibodies to cells was tested in standard flow cytometry procedures as formerly reported [Markel G, Seidman R, Stern N et al (2006). Inhibition of human tumor-infiltrating lymphocyte effector functions by the homophilic carcinoembryonic cell adhesion molecule 1 interactions. J Immunol 177: 6062-6071; Markel G, Seidman R, Cohen Y et al 2009 Feb.; 126(2):186-200. Epub 2008 Jun. 13 Dynamic expression of protective CEACAM1 on melanoma cells during specific immune attack. PBLs are characterized as having small Forward and Side Scatter value in flow cytometry, and were gated accordingly. The cells were stained with a mixture of antibodies, including CD3 (T cell marker), CD56 (NK marker) and an antibody for CEACAM1 (e.g. Kat4c). T cells were defined as CD3-positive CD56-negative lymphocytes (within the Forward & Side Scatter gate described above). NK cells were defined as CD3-negative CD56-positive lymphocytes (within the Forward & Side Scatter gate described above).

### Results

Unusually High Percentage of CEACAM1-Positive NK and T Cells in the Peripheral Blood of Melanoma Patients

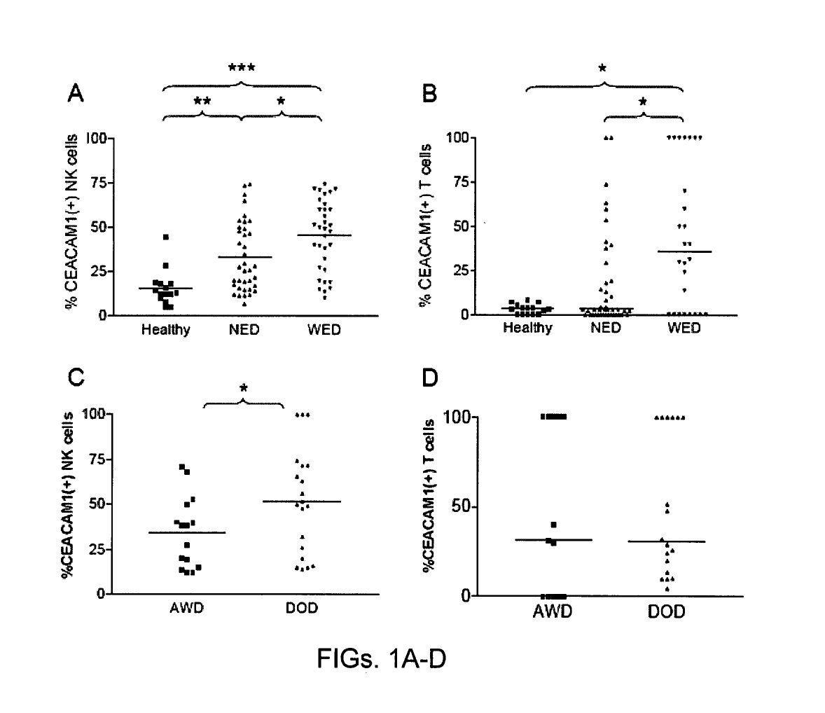

CEACAM1 expression pattern was determined on gated NK and T cells derived from peripheral blood lymphocytes of healthy donors and the melanoma patients. The mean percentage of CEACAM1-positive NK cells in healthy donors was 15% (FIG. 1a). A significantly enhanced proportion of CEACAM-positive NK cells (33%) was observed in NED patients, but the highest proportion (45%) was observed in WED patients (FIG. 1a). Similarly, a significant increase in the mean proportion of CEACAM1-positive T cells (31%) was observed in WED patients (FIG. 1b). The percentage of ceacam+in t cells of NED was lower than 5. A statistically significant positive correlation between CEACAM1 expression by NK and T cells could be observed in WED patients (Spearman's r=0.5, P value<0.05). A similar, yet milder, trend was observed in NED patients, without reaching statistical significance (Spearman's r=0.267, P value=0.082). There was no correlation between percentages of CEACAM1 either on T or NK cells with the serum concentration of CEACAM1. In conclusion, although both soluble CEACAM1 concentrations and CEACAM1 expression on lymphocytes are generally linked to disease activity, they are not connected directly to each other

Indeed, when WED patients were further categorized into DOD (died of disease) and AWD (alive with disease) patients, the mean proportion of CEACAM1-positive NK cells among DOD patients (51.9%) was significantly higher than in AWD patients (34.4%) (FIG. 1c). However, there was no clear correlation between the percentage of CEACAM1 expression and time-to-death among these patients (data not shown). There was no difference in percentage of CEACAM1-positive T cells between DOD and AWD patients (FIG. 1d).

Enhanced CEACAM1 Expression is Functional and Inhibits NK-Mediated Lysis

Peripheral blood lymphocytes were derived either from melanoma patients (exemplar patients 38 and 71) or from healthy donors (FIG. 2a). CEACAM1-mediated inhibition of fresh lymphocytes was tested in natural cytotoxicity assays. The NK-sensitive 721.221 (0.221/Mock) and 721.221 stably transfected with the CEACAM1 protein (0.221/CEACAM1) were used as target cells. Natural killing activity of 0.221 cells was clearly observed with lymphocytes derived from all sources (FIG. 2b). Remarkably, a moderate, yet reproducible and significant inhibition of killing of the 0.221/CEACAM1 cells was observed only with the patient-derived lymphocytes (FIG. 2b). No similar inhibition was measured with the healthy donor derived lymphocytes (FIG. 2b). Similar results were observed with lymphocytes derived from other patients (data not shown) as well as in re-directed lysis experiments performed with concurrent CEACAM1 engagement (data not shown). These results show that enhanced CEACAM1 expression on circulating NK cells is functional, and may expose the patient's immune system to CEACAM1-mediated inhibition.

Sera from Patients do not Induce CEACAM1 Expression on Lymphocytes

Fresh peripheral blood lymphocytes from healthy donors were incubated for 48 hours in culture medium or serum derived either from: healthy donors, patients with low percentage of CEACAM1-positive lymphocytes or patients with high percentage of CEACAM1-positive lymphocytes. CEACAM1 was analyzed on gated lymphocytes cells. There were no significant differences in the expression of CEACAM1 among the different treatments on either CD56(+) or CD56(−) cells (FIG. 2c). These experiments suggest that the high expression of CEACAM1 on lymphocytes observed in melanoma patients is probably not due to systemic soluble factors.

The Phenotype of Circulating NK Cells in Melanoma Patients is Generally Abnormal

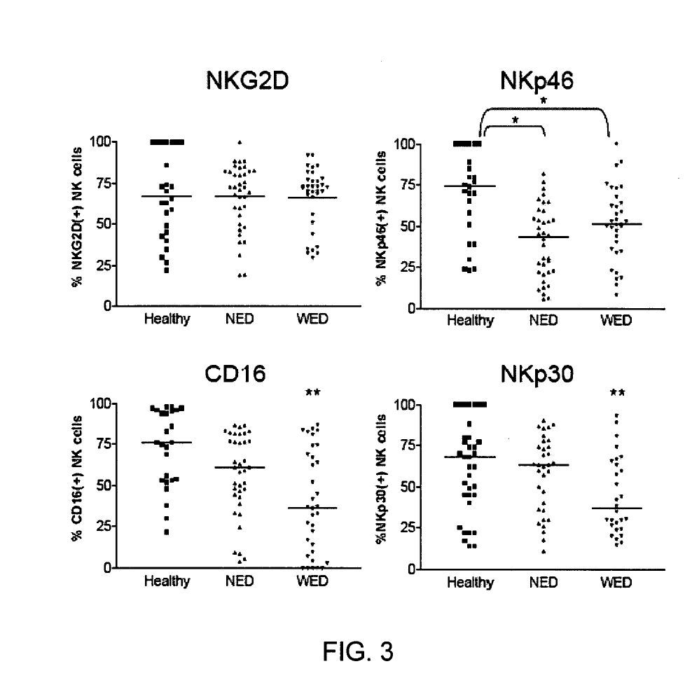

Peripheral blood NK cells were stained for the expression of various killing receptors, including NKG2D, NKp46, CD16 and NKp30. A remarkable decrease was observed in the expression profiles of NKp46, CD16 and NKp30, but not in NKG2D. Specifically, NKp46 was significantly downregulated among all patients, as compared with healthy donors, but there was no significant difference between NED and WED patients. A significant downregulation of CD16 and NKp30 was observed among WED patients, as compared to NED patients and healthy donors. There were no significant differences between NED patients and healthy donors in the expression of these receptors (FIG. 3). A statistically significant positive correlation was identified between CD16, NKp30 and NKp46, and in additional, between NKp46 and NKG2D (Table 1, below). A striking negative correlation was evident between expression of CEACAM1 and the expression of all killing receptors tested, except NKG2D (Table 1). These results indicate on a systemic irregularity in NK cell phenotype, which is not confined only to CEACAM1 expression.

TABLE 1
Correlation between CEACAM1 expression and NK activating
receptors in melanoma patients

| NKG2D | NKp46 | NKp30 | CD16 | CEACAM1 | |
|---|---|---|---|---|---|
| −0.024 | −0.270* | −0.437*** | −0.328** | 1 | CEACAM1 |
| 0.13 | 0.376** | 0.414** | 1 | −0.328** | CD16 |
| 0.072 | 0.353** | 1 | 0.414** | −0.437*** | NKp30 |
| 0.351** | 1 | 0.353** | 0.376** | −0.207* | NKp46 |
| 1 | 0.351** | 0.072 | 0.13 | −0.024 | NKG2D |

The correlation was calculated using Spearman's test. Table summarizes Spearman's R values between each pair of parameters.
*denotes P value <0.05,
**denotes P value <0.01,
***denotes P value <0.001.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A method of diagnosing cancer, the method comprising determining a level of CEACAM1 on isolated peripheral blood lymphocytes (PBLs) of a subject in need thereof, wherein an upregulation of said level of CEACAM1 above a predetermined threshold is indicative of cancer in said subject.

2. The method of claim 1, wherein the cancer is melanoma.

3. The method of claim 1, wherein said PBLs comprise T cells.

4. The method of claim 1, wherein said PBLs comprise NK cells.

5. The method of claim 1, wherein said determining a level of CEACAM1 on isolated peripheral blood lymphocytes (PBLs) is effected by FACS.

6. The method of claim 1, wherein said cancer does not include a hematological cancer.

7. The method of claim 1, further comprising informing the subject on the presence or absence of the cancer or stage thereof.

8. The method of claim 1, further comprising validating the diagnosis or prognosis using a method selected from the group consisting of surgical biopsy, imaging, pathology and molecular testing.

9. The method of claim 1, wherein said determining is effected ex vivo.
